(19) United States
(12) Patent Application Publication
(10) Pub. No.: US 2021/0346553 A1
(43) Pub. Date: Nov. 11, 2021

(54) DISINFECTING SYSTEM AND METHOD

(71) Applicant: SteriGun, Inc., Hutto, TX (US)

(72) Inventor: Dale Quimby PEARSON, Jr., El Cajon, CA (US)

(21) Appl. No.: 17/315,134

(22) Filed: May 7, 2021

Related U.S. Application Data
(60) Provisional application No. 63/021,621, filed on May 7, 2020.

(51) Int. Cl. A61L 2/20; A61L 2/26

(52) U.S. Cl. CPC A61L 2/208; A61L 2/20; A61L 2202/15; A61L 2202/25; A61L 2202/16; A61L 2/26

(57) ABSTRACT

A system can include a delivery manifold with orifice openings arranged asymmetrically. The system can include a pump configured to pressurize a (e.g., disinfecting) fluid to an ultra-high pressure (e.g., 9,000 to 50,000 PSI, 9,000 to 65,000 PSI, any pressure over 9,000 PSI, or any suitable pressure). The pressurized fluid can be expelled through a delivery manifold with asymmetrically arranged orifice openings. In some embodiments, this arrangement can release a dry disinfecting vapor into the air that can disinfect an environment, surface, or object without wetting the item to be disinfected.

### CROSS-REFERENCE TO RELATED APPLICATION

This application claims the benefit of U.S. Provisional Application No. 63/021,621, filed May 7, 2020, which is incorporated by reference herein in its entirety for all purposes.

### FIELD OF THE DISCLOSURE

This relates to a disinfecting system and, more specifically, a disinfecting sprayer system including a delivery manifold with orifice openings arranged asymmetrically

### BACKGROUND OF THE DISCLOSURE

Disinfecting liquids can be used to kill mold, bacteria, and viruses in an environment. Various methods of delivery can be used to apply the disinfectant to the environment or surface to be sterilized. In some situations, a liquid disinfectant can be applied directly to the environment, surface, or object to be sanitized. For example, liquids can be applied using sponges, mops, sprayers, and the like. In some situations, the application of a liquid disinfectant can make the environment, surface, or object wet.

### SUMMARY OF THE DISCLOSURE

This relates to a disinfecting system and, more specifically, a disinfecting sprayer system including a delivery manifold with orifice openings arranged asymmetrically. The disinfecting system can include a pump configured to pressurize a disinfecting fluid to an ultra-high pressure (e.g., 9,000 to 50,000 PSI, 9,000 to 65,000 PSI, any pressure over 9,000 PSI, or any suitable pressure). The pressurized fluid can be expelled through a delivery manifold with asymmetrically arranged orifice openings. In some embodiments, this arrangement can release a dry disinfecting vapor into the air that can disinfect an environment, surface, or object without wetting the item to be disinfected. In some embodiments, the system can be used to deliver other types of fluids, such as irritants (e.g., pepper spray, mace, etc.)

### BRIEF DESCRIPTION OF THE DRAWINGS

FIG. 1 illustrates a block diagram of an exemplary disinfecting system according to some embodiments.

FIG. 2 illustrates an exemplary delivery system according to some embodiments.

FIG. 3 illustrates an exemplary diffuser shroud included in a disinfecting system according to some embodiments.

FIG. 4 illustrates an exemplary delivery manifold according to some embodiments.

### DETAILED DESCRIPTION

In the following description, reference is made to the accompanying drawings which form a part hereof, and in which it is shown by way of illustration specific examples that can be practiced. It is to be understood that other examples can be used and structural changes can be made without departing from the scope of the examples of the disclosure.

This relates to a disinfecting system and, more specifically, a disinfecting sprayer system including a delivery manifold with orifice openings arranged asymmetrically. The disinfecting system can include a pump configured to pressurize a disinfecting fluid to an ultra-high pressure (e.g., 9,000 to 50,000 PSI, 9,000 to 65,000 PSI, any pressure over 9,000 PSI, or any suitable pressure). The pressurized fluid can be expelled through a delivery manifold with asymmetrically arranged orifice openings. In some embodiments, this arrangement can release a dry disinfecting vapor into the air that can disinfect an environment, surface, or object without wetting the item to be disinfected. In some embodiments, the system can be used to deliver other types of fluids, such as irritants (e.g., pepper spray, mace, etc.).

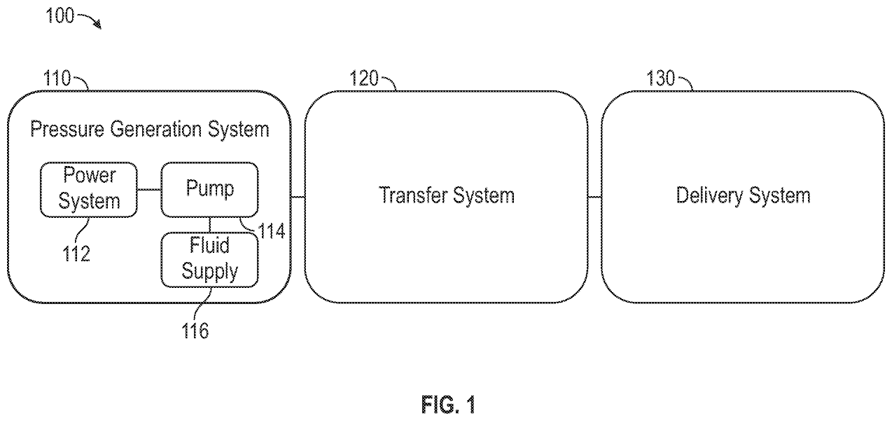

FIG. 1 illustrates a block diagram of an exemplary disinfecting system 100 according to some embodiments. In some embodiments, the disinfecting system 100 is a molecular displacement disinfector unit. The molecular displacement disinfector unit can use ultra-high pressure (e.g., in the range of 9,000 to 50,000 PSI, 9,000 to 65,000 PSI, any pressure over 9,000 PSI, or any suitable pressure) to create disinfecting aerosolized molecules that displace other molecules in the environment to disinfect the environment. In some examples, the system 100 replaces existing air and viral compounds in the environment with a molecular sterilizing spray.

The form factor of disinfecting system 100 can vary. In some embodiments, disinfecting system 100 is a handheld sterilizing gun. In some embodiments, disinfecting system 100 is coupled to a fixed or movable platform, vehicle, or other structure. In some embodiments, disinfecting system is integrated into a building, facility, or maritime vessel. As shown in FIG. 1, the disinfecting system 100 can include a pressure generating system 110, a transfer system 120, and a delivery system 130.

In some embodiments, the pressure generating system 110 includes a power system 112, a pump 114, and a fluid supply 116. Power system 112 optionally includes one or more of a fuel-powered engine or electric motor to power pump 114. In some embodiments, the fluid supply 116 includes a tank or other container that holds a disinfecting liquid, such as a bleach solution, a hydrogen peroxide solution, or any other liquid disinfectant appropriate for the environment to be disinfected. In some embodiments, fluid supply 116 contains a chemical irritant, such as pepper spray or mace, thus rendering the system 100 capable of delivering a chemical irritant. For example, system 100 can be integrated into a building alarm system that can deploy the chemical irritant in response to (e.g., an indication of) intruders entering the building. In some embodiments, pump 114 is a hydraulic pressure pump capable of ultra-high pressures (e.g., pressures in the range of 9,000 to 50,000 PSI, 9,000 to 65,000 PSI, any pressure over 9,000 PSI, or any suitable pressure). Pump 114 is operatively coupled to the power system to enable the power system to power the pump, for example, and pump 114 is fluidly coupled to the fluid supply 116 (e.g., via tubes, fittings, or other suitable fluid couplings) to deliver the pressurized fluid to the transfer system 120. In some embodiments, the pump 114 pressurizes the disinfecting fluid without the use of an additional pump (e.g., an air pump) to increase the pressure in the system 100.

In some embodiments, the transfer system 120 includes a tube (e.g., a steel tube) or hose rated to withstand the pressure created by pump 114. The transfer system 120 can deliver the pressurized disinfecting fluid from the fluid supply 116 to the delivery system 130 while the fluid remains at an ultra-high pressure (e.g., in the range of 9,000 to 50,000 PSI, in the range of 9,000 to 65,000 PSI, any pressure over 9,000 PSI, or any suitable pressure). The length and form factor of transfer system 120 may be widely varied such that sanitizing system 100 can be implemented as a handheld system, a system mounted on a mobile or stationary platform or vehicle, or a system integrated into a building, facility, or maritime vessel.

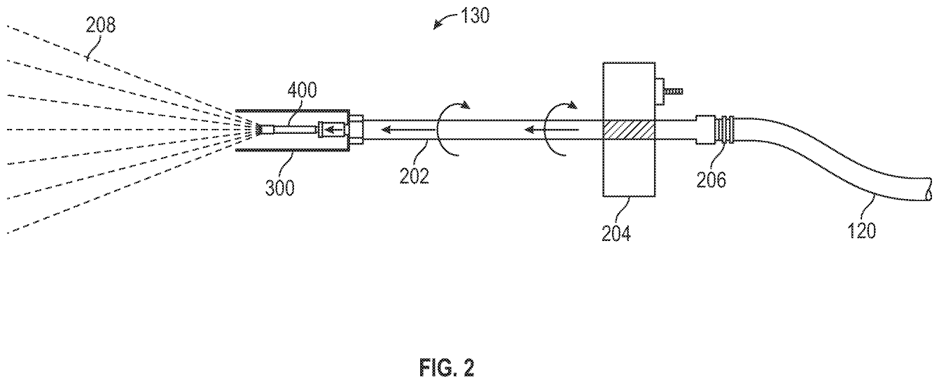

Delivery system 130 may be handheld, mounted on a mobile or stationary platform or vehicle, or integrated into a building, facility, or maritime vessel. In some embodiments, system 100 includes multiple delivery systems 130 (and multiple transfer systems 120 or transfer system 120 includes multiple fluidly coupled conveyances) coupled to pressure generation system 110. Exemplary delivery systems 130 are described in more detail below with reference to FIGS. 2-4.

FIG. 2 illustrates an exemplary delivery system 130 according to some embodiments. In some embodiments, delivery system 130 is included in a disinfecting system, such as system 100 described above with reference to FIG. 1. In some embodiments, delivery system 130 is included in a security system that delivers a chemical irritant (e.g., in response to (e.g., an indication of) an intruder entering a building or vessel with which the system is integrated).

In some embodiments, delivery system 130 includes a transfer shaft or tube 202, an activation area 204, a diffuser shroud 300, and a delivery manifold 400. As shown in FIG. 2, the delivery system 130 is optionally coupled to transfer system 120 by application device connection 206. Although transfer system 120 is illustrated as a hose in FIG. 2, in some embodiments, transfer system 120 is a rigid tube, such as a tube including steel or another suitable material. Application device connection 206 can include a pressure-rated fitting or other coupling configured to deliver the pressurized fluid (e.g., at an ultra-high pressure in the range of 9,000 to 50,000 PSI, 9,000 to 65,000 PSI, any pressure over 9,000 PSI, or any suitable pressure) to the delivery system 130, for example.

In some embodiments, activation area 204 includes a handle or other rated valve to allow the operator of system 100 to control the flow of the fluid to the diffuser shroud 300 and delivery manifold at ultra-high pressure (e.g., in the range of 9,000 to 50,000 PSI, 9,000 to 65,000 PSI, any pressure over 9,000 PSI, or any suitable pressure). In some embodiments, a trigger of the activation area 204 is remotely located. In some embodiments, the transfer shaft or tube 202 may be operatively coupled to an air or electric motor that rotates the transfer shaft or tube during operations. In some embodiments, the transfer shaft or tube 202 does not rotate.

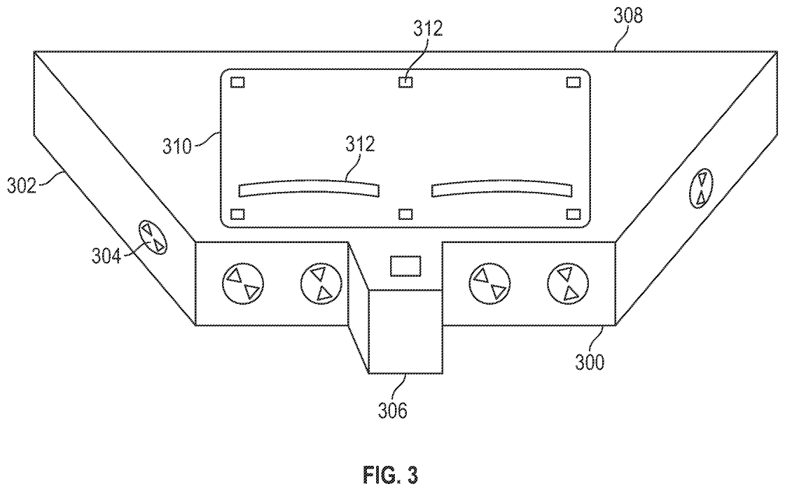

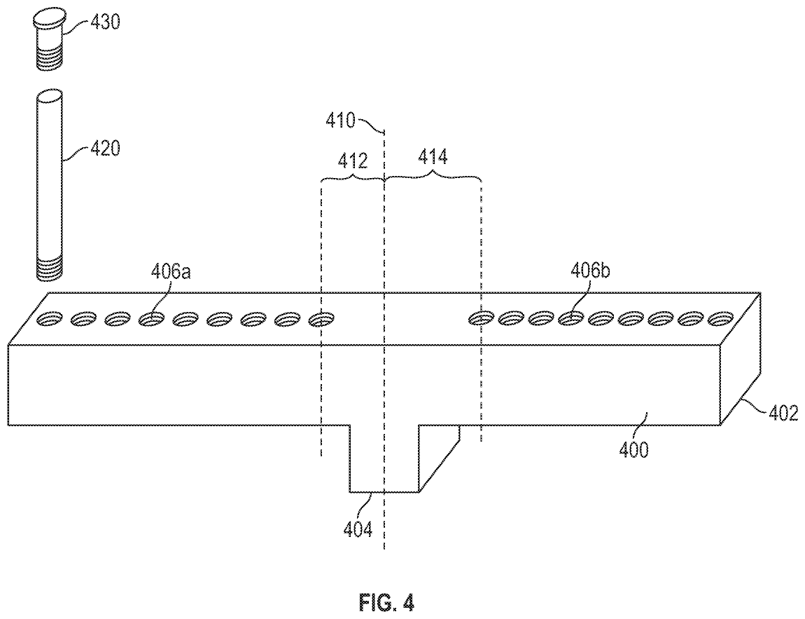

The diffuser shroud 300 and delivery manifold 400 can be coupled to the transfer shaft or tube 200 via a threaded coupling or other suitable fastener, which can therefore provide a mechanism with which to deliver the disinfecting fluid into the surrounding air in the form of a dry vapor. The disinfecting dry vapor can be a high pressure molecular cloud delivered at an ultra-high pressure (e.g., a pressure in the range of 9,000 to 50,000 PSI, 9,000 to 65,000 PSI, any pressure over 9,000 PSI, or any suitable pressure). An exemplary diffuser shroud 300 is described in more detail below with reference to FIG. 3 and an exemplary delivery manifold 400 is described in more detail below with reference to FIG. 4.

FIG. 3 illustrates an exemplary diffuser shroud 300 included in disinfecting system 100 according to some embodiments. As shown in FIG. 2, diffuser shroud 300 can be connected to the transfer shaft or tube 202 of the disinfecting system 100 and disposed surrounding the delivery manifold 400. In some embodiments, the diffuser shroud 300 is configured to shroud the vaporizing orifices of the delivery manifold 400 and provide a manner to control the negative pressure vortex within the delivery system 130 and venturi disruption of cohesive disinfectant molecules expelled by the delivery manifold 400. As will be described in more detail below, the diffuser shroud 300 can be used to adjust the trajectory and disbursement of the (e.g., disinfecting) vapor into the environment. In embodiments with multiple delivery systems 130, the characteristics of the diffuser shrouds 300 of the delivery systems may be varied.

In some embodiments, the diffuser shroud 300 includes a body 302 including adjustable venturi limiters 304. The adjustable venturi limiters 304 are vents through the body 302 of the diffuser shroud 300 that can be adjusted to control the venturi effect of airflow through venturi limiters 300. In some embodiments, the venturi limiters 304 are a fixed size. In some embodiments, the diffuser shroud 300 excludes venturi limiters 304 and body 302 of the diffuser shroud is solid at the illustrated locations of venturi limiters 304. Although diffuser shroud 300 is illustrated as including six venturi limiters 304, in some embodiments, other numbers of venturi limiters are possible. Moreover, in some embodiments, alternate locations of the venturi limiters 304 different from the locations illustrated in FIG. 3 are possible.

In some embodiments, during operation of the disinfecting system 100, the flow of the (e.g., disinfecting) fluid can produce a flow of air from inside the diffuser shroud 300 to outside the diffuser shroud 300 in the direction of the fluid flow. Adjusting the size of the venturi limiters 304 can change the flow of air through the diffuser shroud 300 during operation of disinfecting system, which in turn influences the distance traveled by the disinfecting fluid before aerosolizing and the rate at which the (e.g., disinfecting) fluid aerosolizes.

An interchangeable vortex disruption panel 310 including disruptive air intake locations 312 can be affixed to the body 302 of the diffuser shroud. In some embodiments, vortex disruption panel 310 includes disruptive air intake locations 312 that are open through the body 302 of the diffuser shroud 300 and disrupt pockets of negative pressure within the diffuser shroud created by the flow of the (e.g., disinfecting) fluid expelled by the delivery manifold 400. Other vortex disruption panels 310 are possible, including vortex disruption panels having disruptive air intake locations 312 of different sizes, shapes, or numbers than the disruptive air intake locations 312 illustrated in FIG. 3. In some embodiments, rather than using interchangeable vortex disruption panels 310, the disruptive air intake locations 312 can be directly integrated into the body 302 of the diffuser shroud 300. In some embodiments, diffuser shroud 300 disruptive air intake locations 312 and the body 302 of the diffuser shroud 300 is solid at the locations of air intake locations 312 or the vortex disruption panel 310 itself is solid.

While the disinfecting system 100 is in use, the flow of the sterilizing liquid can create pockets of negative pressure within diffuser shroud 300. These pockets of negative pressure can act to reduce the cohesion of the (e.g., disinfecting) fluid and facilitate breakdown of the fluid into a molecular fog. By changing the vortex disruption panel 310 used with the diffuser shroud 300, the rate at which the (e.g., disinfecting) fluid is aerosolized and the distance at which the (e.g., disinfecting) fluid can travel before becoming aerosolized can be controlled.

In some embodiments, the body 302 of the diffuser shroud 300 includes an attachment end 306 at which the diffuser shroud 300 is coupled to the transfer shaft or tube 202 of the disinfecting system 100 and an outlet end 308 that is open to allow the pressurized disinfecting vapor to be released via the delivery manifold 400.

FIG. 4 illustrates an exemplary delivery manifold 400 according to some embodiments. Delivery manifold 400 can be fluidly coupled to the transfer shaft or tube 202 of the disinfecting system 100 to receive the (e.g., disinfecting) fluid at an ultra-high pressure (e.g., in the range of 9,000 to 50,000 PSI). In some embodiments, disinfecting system 100 includes one or more delivery manifolds 400.

In some embodiments, delivery manifold 400 can include a body 402 having a first plurality of orifice openings 406a, a second plurality of orifice openings 406b, and coupling 404 by which the delivery manifold 400 is attached to the transfer shaft or tube 202 of the disinfecting system 100. The first plurality of orifice openings 406a can be disposed on a first side of the delivery manifold 400 relative to a central axis 410 of the delivery manifold and the second plurality of orifice openings 406b can be disposed on a second side of the delivery manifold 400 relative to the central axis 410 of the delivery manifold. The distance 412 between the center of the innermost of the first plurality of orifice openings 406a and the central axis 410 of the delivery manifold 400 can be different from the distance 414 between the center of the innermost of the second plurality of orifice openings 406b and the central axis of the delivery manifold.

In some embodiments, each orifice opening 406a and 406b is located a unique distance from the central axis 410 of the delivery manifold 400. The asymmetrical arrangement of orifice openings 406a and 406b can allow each fluid stream to be ejected at unique locations as the delivery manifold 400 rotates during use, for example. Therefore, in embodiments in which the delivery manifold 400 rotates during use, each orifice opening 406a and 406b can have a unique circular path around the central axis 410, which can prevent streams from the first plurality of orifice openings 406a and streams from the second plurality of orifice openings 406b from colliding in the air and regaining cohesiveness. In some embodiments, the asymmetrical arrangement of the orifice openings 406a and 406b facilitates rapid vaporization of the (e.g., sanitizing) liquid and production of a dry (e.g., sanitizing) fog. In some embodiments, delivery manifold 400 does not rotate during use. In embodiments in which delivery manifold 400 does not rotate during use, the streams of (e.g., disinfecting) vapor are expelled through the unique locations of orifice openings 406a and 406b.

In some embodiments, the orifice openings 406a and 406b are threaded, thereby allowing an interchangeable orifice 430 to be coupled to each orifice opening of the delivery manifold 430. A variety of interchangeable orifices 430 can be used to control the flow rate, pressure, and other properties of the disinfecting vapor exiting the disinfecting system 100. In some embodiments, orifices can be selected to facilitate flow of the disinfecting fluid in the range of 0.001 to 13 gallons per minute or more than 13 gallons per minute, for example. In some embodiments, the flow rate of the disinfecting fluid through the orifices 430 can be controlled by modifying the pressure exerted by pump 114 or by exchanging the orifices 430 used.

In some embodiments, the interchangeable orifices 430 are coupled to the delivery manifold 400 by a high-pressure molecular attenuator 420. The high-pressure molecular attenuator 420 can be a rigid tube with threaded couplings to attach to the delivery manifold 400 and the orifices 430, for example. In some embodiments, the high-pressure molecular attenuator 420 can provide a straight conveyance through which the fluid is delivered to orifice 430 before being expelled. While traversing high-pressure molecular attenuator 420, the fluid can regain cohesiveness lost while traversing the turns of the delivery manifold 400. In some embodiments, regaining cohesiveness within high-pressure molecular attenuator 420 can enable the disinfecting fluid to travel a further distance before aerosolizing. In some embodiments, the use of high-pressure molecular attenuators 420 can increase consistency in the behavior of the (e.g., disinfecting) fluid from use to use compared to the consistency of using the delivery manifold 400 without high-pressure molecular attenuators 420. In some embodiments, the high-pressure molecular attenuator 420 is eliminated and the interchangeable orifices 430 are instead directly coupled to the orifice openings 406a and 406b of the delivery manifold 400. Removing the high-pressure molecular attenuators 420 can reduce the cohesiveness of the fluid that exits orifices 430 and can reduce the uniformity of the behavior of the fluid. In some embodiments, removing the high-pressure molecular attenuators 420 can decrease bulk of system 100 and increase the speed at which a handheld version of system 100 can be used.

In some embodiments, the size of the openings of the interchangeable orifices 430 and the pressure of the (e.g., disinfecting) fluid can cause the fluid to lose cohesiveness and explode upon exiting the delivery manifold, turning into a (e.g., disinfecting) dry vapor. The interchangeable orifices 430 can be selected based on the desired distance for the (e.g., disinfecting) liquid to travel before aerosolizing: fluids ejected through larger orifice openings will travel further before losing cohesiveness than fluids ejected through smaller orifice openings. In some embodiments, the (e.g., disinfecting) liquid is vaporized by the pressure generated by pump 114 without the use of heightened air pressure within system 100 or the application of heat for the purpose of increasing pressure in the system 100.

In some embodiments, the pressure generation system 110 delivers power, using the power system 112, to pump 114. The pump is able to pressurize fluid contained in fluid supply 116, for example. In some embodiments, the fluid can be any liquid disinfectant. In some embodiments, the fluid can be a chemical irritant. Transfer system 120 can deliver the fluid to delivery system 130. In some embodiments, a diffuser shroud controls the airflow and air pressure in and around the delivery system while delivery manifold 400 ejects the pressurized (e.g., sterilizing) fluid. In some embodiments, the pressurized (e.g., sterilizing) fluid is released into the air of an environment the form of a dry (e.g., disinfecting) cloud. In some embodiments, the sterilized fluid can disinfect objects, air, and surfaces in the environment into which it is released. In some embodiments, while the fluid is being ejected by the delivery system 130, an engine or motor rotates transfer shaft 220 of the system 100. In some embodiments, the delivery system 130 is coupled to the transfer shaft 220 and rotates with the transfer shaft.

In some embodiments, a system includes: a fluid supply tank configured to contain a disinfecting fluid; a pump configured to pressurize the disinfecting fluid; a delivery system fluidly coupled to the pump and configured to expel the disinfecting fluid, the delivery system including a delivery manifold having a plurality of orifice openings disposed in an asymmetrical arrangement. In some embodiments, the delivery manifold includes: a central axis; a first plurality of orifice openings positioned a first distance from the central axis of the delivery manifold; and a second plurality of orifice openings positioned a second distance from the central axis of the delivery manifold. In some embodiments, the delivery manifold includes a central axis, and the delivery manifold is configured to rotate around the central axis during operation of the system. In some embodiments, the delivery manifold is configured to expel the disinfecting fluid in the form of a dry vapor. In some embodiments, the pump pressurizes the disinfecting vapor to a pressure over 9,000 PSI. In some embodiments, the system includes a diffuser shroud disposed around the delivery manifold. In some embodiments, the diffuser shroud includes one or more adjustable venturi limiters and one or more disruptive air intake locations. In some embodiments, the disinfecting liquid includes hydrogen peroxide or bleach.

In some embodiments, a system includes means for containing a disinfecting fluid; means for pressurizing the disinfecting fluid; means for expelling the disinfecting fluid, the means for expelling the disinfecting fluid being fluidly coupled to the means for pressurizing the disinfecting fluid, the means for expelling the disinfecting fluid including a delivery manifold having a plurality of orifice openings disposed in an asymmetrical arrangement. In some embodiments, the means for expelling the disinfecting fluid includes: a central axis; a first plurality of orifice openings positioned a first distance from the central axis of the means for expelling the disinfecting fluid; and a second plurality of orifice openings positioned a second distance from the central axis of the means for expelling the disinfecting fluid. In some embodiments, the means for expelling the disinfecting fluid includes a central axis, and the means for expelling the disinfecting fluid is configured to rotate around the central axis during operation of the system. In some embodiments, the means for expelling the disinfecting fluid is configured to expel the disinfecting fluid in the form of a dry vapor. In some embodiments, the means for pressurizing the disinfecting fluid pressurizes the disinfecting vapor to a pressure over 9,000 PSI. In some embodiments, the system further includes a diffuser shroud disposed around the means for expelling the disinfecting fluid. In some embodiments, the diffuser shroud includes one or more adjustable venturi limiters and one or more disruptive air intake locations. In some embodiments, the disinfecting liquid includes hydrogen peroxide or bleach.

In some embodiments, a delivery manifold includes a central axis; and a body including: a first plurality of orifice openings positioned a first distance from the central axis of the delivery manifold; and a second plurality of orifice openings positioned a second distance from the central axis of the delivery manifold. In some embodiments, the orifice openings include threaded sapphire receives configured to be coupled to an orifice. In some embodiments, the first plurality of orifice openings and the second plurality of orifice openings are configured to expel a pressurized disinfecting fluid in the form of a dry vapor.

Although examples have been fully described with reference to the accompanying drawings, it is to be noted that various changes and modifications will become apparent to those skilled in the art. Such changes and modifications are to be understood as being included within the scope of examples of this disclosure as defined by the appended claims.

1. A system comprising:
a fluid supply tank configured to contain a fluid;
a pump configured to pressurize the fluid;
a delivery system fluidly coupled to the pump and configured to expel the fluid, the delivery system including a delivery manifold having a plurality of orifice openings disposed in an asymmetrical arrangement.

2. The system of claim 1, wherein the delivery manifold includes:
a central axis;
a first plurality of orifice openings positioned a first distance from the central axis of the delivery manifold; and
a second plurality of orifice openings positioned a second distance from the central axis of the delivery manifold.

3. The system of claim 1, wherein:
the delivery manifold includes a central axis, and
the delivery manifold is configured to rotate around the central axis during operation of the system.

4. The system of claim 1, wherein the delivery manifold is configured to expel the disinfecting fluid in the form of a dry vapor.

5. The system of claim 1, wherein the pump pressurizes the fluid to a pressure over 9,000 PSI.

6. The system of claim 1, further comprising a diffuser shroud disposed around the delivery manifold.

7. The system of claim 6, wherein the diffuser shroud includes one or more adjustable venturi limiters and one or more disruptive air intake locations.

8. The system of claim 1, wherein the fluid includes hydrogen peroxide or bleach.

9. A system comprising:
means for containing a fluid;
means for pressurizing the fluid;
means for expelling the fluid, the means for expelling the fluid being fluidly coupled to the means for pressurizing the fluid, the means for expelling the fluid including a delivery manifold having a plurality of orifice openings disposed in an asymmetrical arrangement.

10. The system of claim 9, wherein the means for expelling the fluid includes:
a central axis;
a first plurality of orifice openings positioned a first distance from the central axis of the means for expelling the fluid; and
a second plurality of orifice openings positioned a second distance from the central axis of the means for expelling the fluid.

11. The system of claim 9, wherein:
the means for expelling the fluid includes a central axis, and
the means for expelling the fluid is configured to rotate around the central axis during operation of the system.

12. The system of claim 9, wherein the means for expelling the fluid is configured to expel the fluid in the form of a dry vapor.

13. The system of claim 9, wherein the means for pressurizing the fluid pressurizes the fluid to a pressure over 9,000 PSI.

14. The system of claim 9, further comprising a diffuser shroud disposed around the means for expelling the fluid.

15. The system of claim 14, wherein the diffuser shroud includes one or more adjustable venturi limiters and one or more disruptive air intake locations.

16. The system of claim 9, wherein the fluid includes hydrogen peroxide or bleach.

17. A delivery manifold, comprising:
a central axis; and
a body including:
a first plurality of orifice openings positioned a first distance from the central axis of the delivery manifold; and
a second plurality of orifice openings positioned a second distance from the central axis of the delivery manifold.

18. The delivery manifold of claim 17, wherein the orifice openings include threaded sapphire receives configured to be coupled to an orifice.

19. The delivery manifold of claim 17, wherein the first plurality of orifice openings and the second plurality of orifice openings are configured to expel a fluid in the form of a dry vapor.
